(19) United States
(12) Patent Application Publication
(10) Pub. No.: US 2010/0104546 A1
(43) Pub. Date: Apr. 29, 2010

(54) MODULATORS OF ANTIGEN-DEPENDENT T CELL PROLIFERATION

(75) Inventors: Antonio Ferrante, North Adelaide (AU); Nick Gorgani, Beaumont Hills (AU); Charles S.T. Hii, Marino (AU)

Correspondence Address: BROWN & MICHAELS, PC;400 M & T BANK BUILDING, 118 NORTH TIOGA ST, ITHACA, NY, 14850, US

(73) Assignee: CHILDREN, YOUTH AND WOMEN'S HEALTH SERVICE, North Adelaide (AU)

(21) Appl. No.: 12/525,898

(22) PCT Filed: Feb. 5, 2008
(86) PCT No.: PCT/AU08/00138

§ 371 Date: Jan. 8, 2010

(30) Foreign Application Priority Data
Feb. 5, 2007 (AU) 2007900525

(51) Int. Cl. A61K 35/12; A61K 31/20; C07C 69/02

(52) U.S. Cl. 424/93.71; 514/560; 554/230

(57) ABSTRACT

The present invention relates to a method of inhibiting antigen-dependent proliferation of T-cells in a subject without substantially inhibiting mitogen-dependent proliferation of T-cells in the subject. The method includes administering to the subject an effective amount of a polyunsaturated fatty acid, or a salt or derivative of a polyunsaturated fatty acid.

This application claims priority from Australian provisional patent application No. 2007900525 filed on 5 Feb. 2007, the contents of which are to be taken as incorporated herein by this reference.

### FIELD OF THE INVENTION

The present invention relates to a method of modulating antigen-dependent proliferation of T-cells.

### BACKGROUND OF THE INVENTION

T-cell mediated pathological conditions constitute major areas of unmet medical need. Thus, there is a need for therapeutic agents that regulate T cell activation and the concomitant production of cytokines for management and/or treatment of T-cell mediated pathological conditions.

T lymphocytes (T cells) are an important component of a mammalian immune response. T cells recognize antigens that are associated with a self-molecule encoded by genes within the major histocompatibility complex (MHC). The antigen may be displayed together with MHC molecules on the surface of antigen presenting cells, virus infected cells, cancer cells, grafts, and the like. The T cell system eliminates these altered cells that pose a health threat to the host mammal. T cells include helper T cells (CD4⁺) and cytotoxic T-lymphocytes (CD8⁺). Helper T cells (TH) proliferate extensively following recognition of an antigen-MHC complex on an antigen presenting cell. Helper T cells also secrete a variety of cytokines, such as lymphokines, that play a central role in the activation of B cells, cytotoxic T-lymphocytes, and a variety of other cells that participate in the immune response. Cytotoxic T-lymphocytes are able to cause the destruction of other cells.

A central event in both humoral and cell mediated immune responses is the activation and clonal expansion of helper T cells. Helper T cell activation is initiated by the interaction of the T cell receptor (TCR)-CD3 complex with an antigen-MHC on the surface of an antigen presenting cell. This interaction mediates a cascade of biochemical events that induce the resting helper T cell to enter a cell cycle (the G0 to G1 transition) and results in the expression of a high affinity receptor for IL-2. The activated T cell progresses through the cycle proliferating and differentiating into memory cells or effector cells.

The T-helper cell subsets (Th1 and Th2) define 2 pathways of immunity: cell-mediated immunity and humoral immunity. Release profiles of cytokines for Th1 and Th2 subtypes influence selection of effector mechanisms and cytotoxic cells. Th1 cells, a functional subset of CD4⁺ cells, are characterized by their ability to boost cell-mediated immunity and produce cytokines including Il-2, interferon-gamma, and lymphotoxin beta. IL-2 and interferon-gamma secreted by Th1 cells activate macrophages and cytotoxic cells. Th2 cells are also CD4⁺ cells, but are distinct from Th1 cells. Th2 cells are characterized by their ability to boost humoral immunity, such as antibody production. Th2 cells produce cytokines, including IL-4, IL-5, and IL-10, IL-4, IL-5, and IL-10 secreted by Th2 cells increase production of eosinophils and mast cells, as well as enhance production of antibodies, including IgE, and decrease the function of cytotoxic cells.

Overproduction of cytokines produced by either or both of Th1 and Th2 cells impacts a host of medical disorders. For example, overproduction of Th1 cytokines contributes to pathogenesis of various autoimmune disorders, such as multiple sclerosis and rheumatoid arthritis. Overproduction of Th2 cytokines contributes to pathogenesis of allergic disorders.

CD8⁺ cytotoxic T-lymphocytes (CTLs) are involved in pathogenic destruction of tissue in some autoimmune diseases. For example, CTLs are implicated in destruction of pancreatic beta cells during the course of autoimmune type I diabetes. CTLs also mediate tissue damage associated with graft-versus host disease (GVHD).

The present invention relates to methods of modulating the proliferation of T cells.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention arises from the investigation of engineered derivatives of polyunsaturated fatty acids in antigen-dependent inflammation. In particular, it has been recognized that polyunsaturated fatty acids, and derivatives of the polyunsaturated fatty acids, have the ability to block antigen dependent, but not the mitogen (non-specific) dependent proliferation of T-cells.

The compounds also have other properties including: (i) inhibition of Th1 and Th2 cytokine production (the adaptive immune system) but not cytokines formed by the innate immune system; (ii) reduction in antigen induced inflammation; (iii) promotion of the generation of T suppressor/regulatory cells; (iv) promotion of the generation of long lasting immunosuppressive activity, (v) promotion of immunological tolerance; and promotion of T cell anergy. In addition, the polyunsaturated fatty acids are able to mediate these effects at a low dose, and the effects are long lasting.

Thus, polyunsaturated fatty acids and their derivatives have potential for use in the prevention and/or treatment of antigen-induced inflammation and autoimmune diseases, selective inhibition of Th1 and Th2 cytokine production and prevention and/or treatment of diseases caused by increased production of Th1 and Th2 cytokines, transfer of immunosuppressive activity from donor to recipient, and the promotion of immunological tolerance.

The findings also indicate importantly that the polyunsaturated fatty acids and their derivatives have potential antigen-specific immunosuppression activity rather than global immunosuppression activity.

It has also been found that these effects on sensitized T cells are mediated through the PKC to ERK1/2 signaling pathway.

As such, this finding indicates that agents that modulate the activity of this signaling pathway may be used to generally modulate the responsiveness of sensitized T cells to antigens, and that the ability of agents to modulate the activity of the PKC to ERK1/2 signally pathway can be used to identify new agents with the ability to modulate the responsiveness of T cells to antigens.

The present invention provides a method of inhibiting antigen-dependent proliferation of T-cells in a subject without substantially inhibiting mitogen-dependent proliferation of T-cells in the subject, the method including administering to the subject an effective amount of a polyunsaturated fatty acid, or a salt or derivative of a polyunsaturated fatty acid.

The present invention also provides use of a polyunsaturated fatty acid, or a salt or a derivative thereof, in the preparation of a medicament for inhibiting antigen-dependent proliferation of T-cells in a subject without substantially inhibiting mitogen-dependent proliferation of T-cells in the subject.

The present invention also provides a method of treating a subject susceptible to developing a T-cell mediated disease, condition or state, the method including administering to the subject an effective amount of a polyunsaturated fatty acid, or a salt or derivative thereof.

The present invention also provides use of a polyunsaturated fatty acid in the preparation of a medicament for treating a subject susceptible to developing a T-cell mediated disease, condition or state.

The present invention also provides a method of promoting T cell anergy in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for promoting T cell anergy in a subject:

The present invention also provides a method of reducing the level and/or activity of one or more Th1 and/or Th2 antigen-induced cytokines in a subject without substantially inhibiting the level and/or activity of innate immune cytokines in the subject, the method including administering to the subject an effective amount of a polyunsaturated fatty acid, or a salt or derivative thereof.

The present invention also provides use of a polyunsaturated fatty acid, or a salt or derivative thereof in the preparation of a medicament for inhibiting the level and/or activity of one or more Th1 and/or Th2 antigen-induced cytokines in a subject without substantially inhibiting the level and/or activity of innate immune cytokines in the subject.

The present invention also provides a method of preventing and/or treating antigen-induced inflammation in a subject, the method including administering to the subject an effective amount of a polyunsaturated fatty acid or a salt or derivative thereof.

The present invention also provides use of a polyunsaturated fatty acid, or a salt or derivative thereof, in the preparation of a medicament for preventing and/or treating antigen induced inflammation in a subject.

The present invention also provides a method of increasing the level and/or activity of T suppressor cells in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for increasing the level and/or activity of T suppressor cells in a subject.

The present invention also provides a method of increasing the period and/or level of immunosuppressive activity in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for increasing the period and/or level of immunosuppressive activity in a subject.

The present invention also provides a method of increasing immunological tolerance in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for increasing immunological tolerance in a subject.

The present invention also provides a method of increasing immunosuppressive activity in a subject, the method including:
exposing T cells isolated from a subject to an effective amount of a polyunsaturated fatty acid, or a salt or derivative thereof; and
introducing the T cells so exposed back into the subject.

The present invention also provides use of T-cells isolated from a subject and treated with a polyunsaturated fatty acid, or a salt or derivative thereof, in the preparation of a medicament for increasing immunosuppressive activity in the subject when introduced into back into the subject.

The present invention also provides a method of treating a subject susceptible to developing an autoimmune disease, the method including administering to the subject an effective amount of a polyunsaturated fatty acid, or a salt of derivative thereof.

The present invention also provides use of a polyunsaturated fatty acid, or a salt or a derivative thereof, in the preparation of a medicament for treating a subject susceptible to developing an autoimmune disease.

The present invention also provides a method of reducing rejection of a transplanted organ in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for reducing rejection of a transplanted organ in a subject.

The present invention also provides a method of reducing graft versus host disease in a subject, the method including administering to the subject an effective amount of a polyunsaturated fatty acid, or a salt or derivative thereof.

The present invention also provides use of a polyunsaturated fatty acid, or a salt or derivative thereof, in the preparation of a medicament for reducing graft versus host disease.

The present invention also provides a method of inhibiting antigen-dependent proliferation of T-cells in a subject, the method including administering to the subject a polyunsaturated fatty acid, or a salt or derivative thereof, at a concentration of less than 10 mg/kg body weight.

The present invention also provides a pharmaceutical dosage form for administration to a subject, the dosage, form including less than 10 mg/kg body weight of a polyunsaturated fatty acid, or a salt or derivative thereof.

The present invention also provides a method of preventing and/or treating a T-cell mediated disease, condition or state in a subject, the method including administering to the subject a polyunsaturated fatty acid, or a salt or derivative thereof, at a concentration of less than 10 mg/kg body weight.

The present invention also provides a method of reducing the level and/or activity of one or more Th1 and/or Th2 antigen-induced cytokines in a subject, the method including administering to the subject a polyunsaturated fatty acid, or a salt or derivative thereof, at a concentration of less than 10 mg/kg body weight.

The present invention also provides a method of preventing and/or treating an auto-immune disease in a subject, the method including administering to the subject a polyunsaturated fatty acid, or a salt or derivative thereof, at a concentration of less than 10 mg/kg body weight.

The present invention also provides a method of modulating responsiveness of a sensitised T cell to an antigen, the method including modulating activity of one or more signalling pathways in the T cell.

The present invention also provides a method of modulating T cell anergy, the method including modulating the activity of the ERK1/2 signalling pathway in the T cell.

The present invention also provides a method of promoting T cell anergy in a subject, the method including administering to the subject an agent that inhibits the activity of the ERK1/2 signalling pathway in a T cell.

The present invention also provides a method of increasing the period and/or level of immunosuppressive activity in a subject, the method including administering to the subject an effective amount of an agent that inhibits the activity of the ERK1/2 signalling pathway in a T cell.

The present invention also provides a method of increasing immunological tolerance in a subject, the method including administering to the subject an effective amount of an agent that inhibits the activity of the ERK1/2 signalling pathway in a T cell.

The present invention also provides a method of identifying an agent that promotes T cell anergy, the method including identifying an agent that inhibits the activity of the ERK1/2 signalling pathway in a T cell, wherein an agent that inhibits the ERK1/2 signalling pathway is a candidate agent for promoting T cell anergy.

The present invention also provides a method of increasing the period and/or level of immunosuppressive activity in a subject, the method including administering to the subject an effective amount of an agent that inhibits the ERK1/2 signalling pathway in a T cell.

The present invention also provides a method of increasing immunological tolerance in a subject, the method including administering to the subject an effective amount of an agent that inhibits the ERK1/2 signalling pathway in a T cell.

The present invention also provides a method of identifying an agent that promotes T cell anergy, the method including identifying an agent that inhibits the ERK1/2 signalling pathway in a T cell, wherein an agent that inhibits the ERK1/2 signalling pathway is a candidate agent for promoting T cell anergy.

Various terms that will be used throughout the specification have meanings that will be well understood by a skilled addressee. However, for ease of reference, some of these terms will now be defined.

The term “polyunsaturated” as used throughout the specification is to be understood to mean a molecule including a carbon chain which contains more than one double and/or triple valence bond. The term includes within its scope geometric isomers.

The term “polyunsaturated fatty acid” as used throughout the specification is to be understood to mean a carboxylic acid, or a salt thereof, the carboxylic acid including a carbon chain of which contains more than one double and/or triple valence bond.

In this regard, a derivative of a polyunsaturated fatty acid is to be understood to mean a compound which has the property of inhibiting antigen-dependent proliferation of T-cells and (i) has a covalent attachment of one or more atoms to the carboxylic acid, for example, an amino acid attached to the carboxylic acid group; or (ii) is an acyl derivative of the polyunsaturated fatty acid; and or (iii) has a replacement of the carboxylic acid group with a functional group, such as a polyunsaturated nitroalkene or a nitroalkyne.

It will also be appreciated that the compounds include a pro-drug of the compounds, being a precursor which upon administration to a biological system, undergoes chemical conversion by metabolic or chemical processes to yield a polyunsaturated fatty acid of the present invention, or a salt and/or a derivative thereof.

It will further be appreciated that the polyunsaturated fatty acids of the present may also be optionally substituted. The term “substituted” means that a hydrogen atom on a molecule has been replaced with a different atom or molecule. The atom or molecule replacing the hydrogen atom is denoted as a “substituent.” The term substituted specifically envisions and allows for substitutions that are common in the art. However, it is generally understood by those skilled in the art that the substituents should be selected so as to not adversely affect the pharmacological characteristics of the compound or adversely interfere with the use of the medicament.

The substituent groups may be independently selected from the group consisting of: halogen (F, Cl, Br, I), ═O, ═S, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkyl, heteroarylalkyl, cycloalkylalkyl, heterocycloalkylalkyl, arylalkyloxy, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, cyano, nitro, amino, thio, thioalkyl, carboxyl, carboxyl ester, amido, keto, acyl, —NHCOO—, —NHCONH—, and —C(═NOH)—. Where appropriate, the substituent group may be a terminal group or a bridging group. The substituent group may comprise two or more of the aforementioned groups bonded to one another.

In this regard, the term “alkyl” as used throughout the specification is to be understood to mean a group or part of a group of saturated straight chain, branched or cyclic hydrocarbon groups, such as a C₁-C₄₀ alkyl, a C₁-C₃₀ alkyl, or a C₁-C₆ alkyl. Examples of straight chain and branched alkyl include methyl, ethyl, propyl, isopropyl, butyl, sec-butyl, tert-butyl, n-pentyl and branched isomers thereof, n-hexyl and branched isomers thereof, n-heptyl and branched isomers thereof, n-octyl and branched isomers thereof, n-nonyl and branched isomers thereof, and n-decyl and branched isomers thereof. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl, and the like. An alkyl group may be further optionally substituted by one or more optional substituents as herein defined.

The term “alkenyl” as used throughout the specification is to be understood to mean a group or part of a group straight chain, branched or cyclic, hydrocarbon residues containing at least one carbon to carbon double bond including ethylenically mono-, di- or poly-unsaturated alkyl or cycloalkyl groups. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4, pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, 1,3,5,7-cyclooctatetraenyl, and the like. An alkenyl group may be optionally substituted by one or more optional substituents as herein defined.

The term “alkynyl” as a group or part of a group as used throughout the specification is to be understood to mean straight chain, branched or cyclic hydrocarbon residues containing at least one carbon-carbon triple bond including ethynically mono-, di- or poly-unsaturated alkyl or cycloalkyl groups as previously defined. Examples include ethynyl, 1-propynyl, 2-propynyl, butynyl isomers, pentynyl isomers, and the like. An alkynyl group may be further optionally substituted by one or more optional substituents as herein defined.

The term “heterocyclyl” as a group or part of a group as used throughout the specification is to be understood to mean monocyclic, polycyclic, fused or conjugated hydrocarbon residues wherein one or more carbon atoms (and where appropriate, hydrogen atoms attached thereto) are replaced by a heteroatom so as to provide a non-aromatic residue. Suitable heteroatoms include nitrogen, oxygen, sulphur and selenium. Where two or more carbon atoms are replaced, this may be by two or more of the same heteroatom or by different heteroatoms. Examples of heterocyclic groups include pyrrolidinyl, pyrrolinyl, piperidyl, piperazinyl, morpholino, indolinyl, imiazolidinyl, pyrazolidinyl, thiomorpholino, dioxanyl, tetrahydrofuranyl, tetrahydropyranyl, tetrahydropyrrolyl, and the like. A heterocyclyl group may be further optionally substituted by one or more optional substituents as herein defined.

The term “aryl” as a group or part of a group as used throughout the specification is to be understood to mean: (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; and (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl.

The term “heteroaryl” as a group or part of a group as used throughout the specification is to be understood to mean an optionally substituted aromatic ring having one or more heteroatoms as ring atoms in the aromatic ring with the remainder of the ring atoms being carbon atoms. Suitable heteroatoms include nitrogen, oxygen, sulphur and selenium. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, triazine, tetrazole, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl.

The term “acyl” as a group or part of a group as used throughout the specification is to be understood to mean a group containing the moiety C═O (and not being a carboxylic acid, ester or amide). Examples of acyl include formyl; straight chain or branched alkanoyl such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl.

The terms alkoxy, alkenoxy alkynoxy, aryloxy, heteroaryloxy, heterocyclyloxy and acyloxy respectively denote alkyl, alkenyl, alkynyl, aryl, heteroaryl, heterocyclyl and acyl groups as hereinbefore defined when linked by oxygen.

The term thioalkyl refers to an alkyl group when linked by sulfur.

The term “carboxyl” as a group or part of a group refers generally to the group CO₂H (or a salt thereof) and “carboxyl ester” as a group or part of a group refers generally to the group CO₂R wherein R is any group not being H.

The term “amino” as a group or part of a group as used throughout the specification is to be understood to mean the group NRR′ and “amido” as a group or part of a group refers generally to the group CONRR′, wherein R and R′ can independently be H, alkyl, alkenyl, alkynyl, aryl, acyl, heteroaryl, heterocyclyl, or derivatives thereof.

The term “halo” as used throughout the specification is to be understood to mean a halogen group, including fluoro, chloro, bromo and iodo groups.

The term “subject” as used throughout the specification is to be understood to mean a human or animal subject. In this regard, it will be understood that the present invention includes within its scope veterinary applications. For example, the animal subject may be a mammal, a primate, a livestock, animal (eg. a horse, a cow, a sheep, a pig, or a goat), a companion animal (eg. a dog, a cat), a laboratory test animal (eg. a mouse, a rat, a guinea pig, a bird, a rabbit), an animal of veterinary significance, or an animal of economic significance.

### BRIEF DESCRIPTION OF THE FIGURES

FIG. 1 shows suppression of antigen-dependent splenocyte proliferation by β-oxa-21:3n-3. Mice were immunized with SRBC for 5 days. β-oxa-21:3n-3 was delivered in DMSO and injected intraperitoneally at the indicated doses. After 2 h spleens were removed and single cell suspensions were prepared. The splenocytes were cultured for 3 days in the presence (□), or absence (▪) of SRBC. Thymidine incorporation for final 16 hours of culture was determined. The data is expressed as mean±SEM of 3 separate experiments each in triplicate.

FIG. 2 shows the effect of route of delivery of β-oxa-21:3n-3. Mice were immunized with SRBC for 5 days. Then β-oxa-21:3n-3 dissolved in syngeneic mouse serum and injected intraperitoneally (A), intravenously (B) or gavaged/orally (C) at the indicated concentrations. After 2 h spleens were removed, single cell suspensions prepared and the splenocytes cultured for 3 days in the presence (▪), or absence (□) of SRBC. Thymidine incorporation for final 16 hours of culture was determined. The data is presented as the mean±SEM of 3 separate experiments separate experiments, each in quintuplets.

FIG. 3 shows the effect of β-thia-21:3n-3 on antigen induced ex vivo lymphocyte responses. FIG. 3(A) shows the effect of β-thia-21:3n-3 on antigen-(SRBC) induced ex vivo lymphocyte responses. Mice were immunized with SRBC. After 5 days they were given β-thia-21:3n-3 at the indicated doses i.p. After 2 hours the spleens were removed and processed into single cell suppressions. These were then cultured in the presence (□), or absence of (▪) of SRBC to assess the proliferation. The results are expressed as mean±SEM of 3 experiments. (B) Effect of β-oxa-21:3n-3 on tetanus toxoid (TT)-induced ex vivo lymphocyte responses. Mice were immunized with TT subcutaneously and after 7 days were given β-oxa-21:3n-3 in mouse serum, intraperitoneally. Two hours later the spleen removed, single cell suspension prepared and examined for lymphoproliferation responses to TT in 5 day cultures. The results are expressed as mean±SEM of 3 experiments.

FIG. 4 shows the effect of β-oxa-21:3n-3 on PHA/PMA- or PMA/A23187-stimulated cell adhesion and proliferation. (A) β-oxa-21:3n-3 or vehicle was injected intraperitoneally to 5 day SRBC-sensitized mice. After 2 h splenocytes (1×10⁶ cells) were cultured in a 24 well tissue culture plate (Nunc, Inc. Denmark) in the absence or presence of PHA (2 μg/ml) and PMA (10 ng/ml) for 2 h. Cells were fixed with 2% Para formaldehyde and inspected under a light microscopy and photographed. (B) β-oxa-21:3n-3 or vehicle was injected intraperitoneally or intravenously to 5 day SRBC-sensitized mice. After 2 h splenocytes (0.2 million) were cultured in a 96 well tissue culture plate (Nunc, Inc. Denmark) in the presence of PHA and PMA (PHA/PMA) or PMA (10 ng/ml) and A23187 (1 mM) (PMA/A23187) for 3 days. Thymidine incorporation for final 16 hours of culture was determined. The data represents the mean±SEM of 3 separate experiments each conducted in quintuplets.

FIG. 5(A) shows comparison of the effects of β-oxa-21:3n-3 on human lymphocyte responses, induced by antigen (TT) or mitogen (PHA). The results are presented as mean±SEM of three experiments. (B) Cells were incubated with β-oxa 21:3n-3 (20 μM) for 30 min and then stimulated with PHA-PMA for another 30 min. Lipids were extracted and processed as described in Materials and Methods. Data are presented as mean±SEM (n=4). β-oxa 21:3n-3 incorporation into DAG was compared between unstimulated and stimulated cells using a two-tailed Student's paired t test. Significance of difference, ***, p<0.0001.

FIG. 6 shows the effect of β-oxa-21:3n-3 on ex vivo cytokine production by antigen-stimulated splenocytes. Indicated doses of β-oxa-21:3n-3 or vehicle were injected intraperitoneally to mice 5 days after SRBC-sensitized them. After 2 h spleens were removed, and splenocyte single cell suspensions prepared and cultured in the presence or absence of SRBC. After 3 days in culture cell culture fluids supernatants were collected and stored frozen at −70° C. until use for cytokine measurement. The levels of Th1 and Th2 and macrophage-related cytokines were measured using a BD cytokine array according to manufacturer instructions. The baseline cytokine levels in supernatants of cells proliferating in the absence of SRBC were subtracted from those in the presence of SRBC and plotted against administered β-oxa-21:3n-3 dose. The data represent the mean±SEM of 3 separate experiments each conducted in quintuplets.

FIG. 7 shows the effect of β-oxa-21:3n-3 on cytokine levels of mitogen-stimulated splenocytes. Mice were sensitized with SRBC and after 5 days were injected with β-oxa-21:3n-3 or vehicle intraperitoneally. After 2 h spleens were removed, cell suspensions prepared and incubated in the presence of mitogens PHA/PMA or PMA/A23187. After 3 days of incubation, the culture fluids and were harvested and used to determine the levels of Th1 and Th2 cytokines. Baseline subtracted levels of cytokines plotted in the presence of vehicle or β-oxa-21:3n-3. The values represent the mean±SEM separate experiments each conducted in quintuplets.

FIG. 8 shows the duration of β-oxa-21:3n-3 efficacy and evaluation of cell type affected by the fatty acid. (A) β-oxa-21:3n-3 (80 mg/kg) or vehicle was injected once intraperitoneally to 5 days SRBC-sensitized mice. After the indicated time period spleens were removed and cell suspensions were incubated in the presence or absence of SRBC for 3 days. Thymidine incorporation for final 16 hours of culture was determined. Percent inhibition of proliferation by β-oxa-21:3n-3 compared to vehicle injected animals are depicted against duration of exposure. (B) β-oxa-21:3n-3 (80 mg/kg) or vehicle was injected once intraperitoneally to 5 days SRBC-sensitized mice. After two hours spleens were removed and T cell and MHC II positive cells were enriched using MACS magnetic beads. As indicated, vehicle and β-oxa-21:3n-3 injected animal's enriched cells were then co-cultured in the presence or absence of SRBC for 3 days. Thymidine incorporation for final 16 h of culture was determined. Splenocytes proliferation is plotted versus the type of co-cultured cells.

FIG. 9 shows the effect of β-oxa-21:3n-3 on spleen cell composition and percentage of cells enriched by magnetic beads. (A) Depicts the number of indicated surface marker-bearing cells in the spleen preparations from vehicle (□) or β-oxa-21:3n-3 (▪) administered animals. (B) Represent the number of cells recovered after enrichment by MHC II- and Thy1.2-magnetic beads of β-oxa-21:3n-3 (□) or vehicle (▪) administered animal's splenocytes. Error bars represent±SEM for 3 separate experiments (n=5).

### GENERAL DESCRIPTION OF THE INVENTION

The present invention arises from the investigation of polyunsaturated compounds in antigen-dependent and independent proliferation of T-cells.

In particular, the present studies have used polyunsaturated fatty acids having an oxa or thia substitution at the β position as exemplary polyunsaturated fatty acids. In this regard, the present studies demonstrate that these polyunsaturated fatty acids have the ability to block the antigen dependent, but not mitogen dependent, proliferation of T-cells.

These compounds also have a number of additional characteristic, including: (i) inhibition of Th1 and Th2 cytokine production (the adaptive immune system) but not cytokines formed by the innate immune system; (ii) reduction in antigen induced inflammation; (iii) promotion of the generation of T suppressor/regulatory cells; (iv) promotion of the generation of long lasting immunosuppressive activity, including increasing the period and/or level of immunosuppressive activity; (v) promotion of immunological tolerance, including increasing the period and/or level of immunological tolerance; and promotion of T cell anergy.

Accordingly, in one embodiment the present invention provides a method of inhibiting antigen-dependent proliferation of T-cells in a subject without substantially inhibiting mitogen-dependent proliferation of T-cells in the subject, the method including administering to the subject an effective amount of a polyunsaturated fatty acid, or a salt or derivative of a polyunsaturated fatty acid.

The subject in the various embodiments of the present invention may a human or animal subject.

In one embodiment, the antigen dependent proliferation is inhibited by greater than 2 fold over the mitogen dependent proliferation. In one specific embodiment, the antigen dependent proliferation is inhibited by greater than 4 fold over the mitogen dependent proliferation. In a specific embodiment, the antigen dependent proliferation is inhibited by greater than 10 fold over the mitogen dependent proliferation. In a further embodiment, the antigen dependent proliferation is inhibited by greater than 15 fold over the mitogen dependent proliferation.

In one embodiment, the polyunsaturated compounds of the present invention are polyunsaturated fatty acids including an oxa and/or thia substitution at either or both of the β and γ position of the polyunsaturated fatty acid.

In another embodiment, the derivative of a polyunsaturated fatty acid is a polyunsaturated fatty acid covalently coupled to an amino acid.

In another embodiment, the derivative of a polyunsaturated fatty acid is a polyunsaturated nitroalkene or nitroalkyne.

In another embodiment, the present invention provides use of a polyunsaturated fatty acid, or a salt or a derivative thereof, in the preparation of a medicament for inhibiting antigen-dependent proliferation of T-cells in a subject without substantially inhibiting mitogen-dependent proliferation of T-cells in the subject.

It has also been recognised that the polyunsaturated compounds of the present invention have the capacity for use in treating a subject susceptible to developing a T-cell mediated disease, condition or state, so as to prevent the development of the T-cell mediated disease, condition or state in the subject, or ameliorate the T-cell mediated disease, condition or state that develops in the subject.

Examples of T-cell mediated diseases, conditions or states in the various embodiments of the present invention include an allergic disease, including vasculitis, allergic contact dermatitis and contact dermatoconjunctivitis; a chronic inflammatory disease, including Crohn's disease, inflammatory bowel disease and polymyositis; recurrent inflammatory disease including herpes simplex stromal keratitis; transplant rejection; graft vs. host disease; an autoimmune disease including scleroderma, rheumatoid arthritis, type I diabetes and multiple sclerosis.

In this regard, the utility of the polyunsaturated compounds of the present invention for preventing or ameliorating the development of T cell mediated diseases, conditions or states was unexpected, as the mechanisms underlying the development of such diseases differ in many ways from the mechanisms operating before such diseases are established.

Accordingly, in another embodiment the present invention provides a method of treating a subject susceptible to developing a T-cell mediated disease, condition or state, the method including administering to the subject an effective amount of a polyunsaturated fatty acid, or a salt or derivative thereof, and thereby treat the subject susceptible to developing a T-cell mediated disease, condition or state.

In another embodiment, the present invention provides use of a polyunsaturated fatty acid in the preparation of a medicament for treating a subject susceptible to developing a T-cell mediated disease, condition or state.

In one embodiment, the present invention further includes screening the subject to identify the subject as being susceptible to developing a T-cell mediated disease, condition or state.

Suitable methods of screening include one or more of a genetic assay, a hybridization based assay, an immunological assay, a cell based assay, and a biochemical assay. Such methods are known in the art.

In one embodiment, the T-cell mediated disease, condition or state is an autoimmune disease.

Accordingly, in another embodiment the present invention provides a method of treating a subject susceptible to developing an autoimmune disease, the method including administering to the subject an effective amount of a polyunsaturated fatty acid, or a salt of derivative thereof, and thereby treat the subject susceptible to developing an autoimmune disease, condition or state.

In another embodiment, the present invention provides use of a polyunsaturated fatty acid, or a salt or derivative thereof, in the preparation of a medicament for treating a subject susceptible to developing an autoimmune disease.

In one embodiment, the T-cell mediated disease, condition or state is rejection of a transplant.

Accordingly, in another embodiment the present invention provides a method of reducing rejection of a transplanted organ in a subject, the method including administering to the subject an effective amount of a polyunsaturated fatty acid, or a salt or derivative thereof.

In another embodiment, the present invention provides use of a polyunsaturated fatty acid, or a salt or derivative thereof, in the preparation of a medicament for reducing rejection of a transplanted organ in a subject.

In another embodiment, the T-cell mediated disease, condition or state is graft versus host disease.

Accordingly, in one embodiment the present invention provides a method of reducing graft versus host disease in a subject, the method including administering to the subject an effective amount of a polyunsaturated fatty acid, or a salt or derivative thereof.

In another embodiment, the present invention provides use of a polyunsaturated fatty acid, or a salt or derivative thereof, in the preparation of a medicament for reducing graft versus host disease.

The polyunsaturated fatty acids of the present invention include pharmaceutically acceptable derivatives, salts, solvates, tautomers or pro-drugs thereof.

In one embodiment, the polyunsaturated fatty acid, or a salt or derivative thereof, has 16 to 26 carbon atoms. In one specific embodiment, the polyunsaturated fatty acid, or a salt of derivative thereof, has 18 to 22 carbon atoms.

In one embodiment, the polyunsaturated fatty acid, or a salt or derivative thereof, may have 1 to 6 carbon double bonds, such as having 3, 4, 5 or 6 carbon double bonds.

In another embodiment, the polyunsaturated fatty acid, or a salt or derivative thereof, is an n-3 to n-6 fatty acid.

In one embodiment, the polyunsaturated fatty acid, or a salt or derivative thereof, includes one or more substitutions selected from the group consisting of a hydroxyl, a hydroperoxy, a peroxy, and a carboxyalkyl (eg carboxymethyl) substitution.

In the embodiment in which the polyunsaturated fatty acids for use in the present invention have an oxa and/or thia substitution at either or both of the β and γ position of the polyunsaturated fatty acid, these compounds may be a β-oxa polyunsaturated fatty acid; a β-thia polyunsaturated fatty acid; a γ-oxa polyunsaturated fatty acid; a γ-thia polyunsaturated fatty acid; a β-oxa, γ-oxa polyunsaturated fatty acid; a β-thia, γ-oxa polyunsaturated fatty acid; a β-oxa, γ-thia polyunsaturated fatty acid; or a β-thia, γ-thia polyunsaturated fatty acid.

In one embodiment, the polyunsaturated fatty acid, or a salt or derivative thereof, includes one or more substitutions selected from the group consisting of a hydroxyl, a hydroperoxy, a peroxy, and a carboxyalkyl (eg carboxymethyl) substitution.

Methods for producing such fatty acids are known in the art, for example as described in international patent application WO 96/11908.

Examples of β-oxa compounds include β-oxa-23:4n-6; β-oxa-21:3n-6; β-oxa-21:3n-3; β-oxa-25:6n-6; β-oxa-21:4n-3; 16-OH-β-oxa-21:3n-6; 16-OH-β-oxa-21:3n-3; β-oxa-18:3n-3, β-oxa-20:4n-6, β-oxa-20:5n-3, β-oxa-22:6n-3, β-oxa-23:4n-6, 15-OOH-β-oxa-20:4n-6, β-oxa-23:4n-6, β-oxa-21:3n-6, β-oxa-21:3n-3, β-oxa-25:6n-3, β-oxa-21:4n-3, 16-OH-β-oxa-21:3n-6, 16-OH-β-oxa-21:3n-3.

Examples of β-thia compounds include β-thia-21:3n-6; β-thia-21:3n-3; β-thia-25:6n-3; β-thia-23:4n-6; α-carboxymethyl-β-thia-23:4n-6.

Examples of γ-thia polyunsaturated fatty acids include γ-thia-22:3n-6; γ-thia-22:3n-3; γ-thia-24:4n-6; γ-thia-25:6n-3.

In the embodiment in which the derivative of a polyunsaturated fatty acid is a polyunsaturated fatty acid covalently coupled to an amino acid, in one embodiment the polyunsaturated fatty acid is coupled to the amino acid by way of an amide linkage, although it will be appreciated that the amino acid may be coupled to carboxylic by other means known in the art. Such compounds are generally described in U.S. Pat. No. 5,998,476.

The amino acid may be a natural amino acid, or an amino acid sequence modified either by natural processes, such as post-translational processing, or by a chemical modification technique known in the art. Naturally occurring amino acids include alanine, arginine, asparagine, aspartic acid, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In one embodiment, the amino acid is glycine or aspartic acid.

In one embodiment, the polyunsaturated fatty acid coupled to an amino acid has 16 to 26 carbon atoms. In one specific embodiment, the polyunsaturated fatty acid coupled to an amino acid has 18 to 22 carbon atoms.

In one embodiment, the polyunsaturated fatty acid coupled to an amino acid may have 1 to 6 carbon double bonds, such as having 3, 4, 5 or 6 carbon double bonds.

In another embodiment, the polyunsaturated fatty acid coupled to an amino acid is an n-3 to n-6 fatty acid.

In one embodiment, the compound is γ-linolenic acid-glycine, α-linolenic acid-glycine, arachidonic acid-glycine, docosahexaenoic acid-glycine, eicosapentaenoic glycine, γ-linolenic acid-aspartic acid, α-linolenic acid-aspartic acid, arachidonic acid-aspartic acid, eicosapentaenoic acid-aspartic acid and docosahexaenoic acid-aspartic acid.

In the case of the embodiment in which the derivative of the polyunsaturated fatty acid is a polyunsaturated nitroalkenes or nitroalkynes, in one embodiment the compound has the formula X—NO₂, wherein X is an unsaturated hydrocarbon chain of 14 to 26 carbon atoms, and which may be optionally substituted.

In another embodiment, the compound has a formula of R₁—X—NO₂, wherein X is an unsaturated hydrocarbon chain of 14 to 26 carbon atoms, and which may be optionally substituted, and R₁ is (CH2)ₙ(COOH)ₘ in which n is 0 to 2, and m is independently 0 to 2. Such compounds are as generally described in international patent application WO 01/21575.

In one embodiment, X is a hydrocarbon chain of 18 to 22 carbon atoms, and in one specific embodiment has 3-6 double bonds.

In one embodiment, the compound has an unsaturated hydrocarbon chain having 18 carbon atoms and three double bonds separated by methylene groups, with the first double bond relative to the omega carbon atom being between the third and fourth or sixth and seventh carbon atoms.

In one embodiment, the compound is selected from the group consisting of (z,z,z)-1-Nitro-9,12,15-octadecatriene, (z,z,z)-1-Nitro-6,9,12-octadecatriene, (all-z)-1-Nitro-5,8,11,14-eico-satetraene, (all-z)-1-Nitro-4,7,10,13,16,19-docosahexaene, (all-Z)-4-Nitrotricosa-8,11,14,17-tetraenoic acid, 3-[(all-Z)-Nonadeca-4,7,10,13-tetraenyl]-3-nitropentane-1,5-dicarboxylic acid.

In one embodiment, the polyunsaturated fatty acid, or a salt or derivative thereof, includes one or more substitutions selected from the group consisting of a hydroxyl, a hydroperoxy, a peroxy, and a carboxyalkyl (eg carboxymethyl) substitution.

The administration of the polyunsaturated fatty acid, or a salt or derivative thereof, to the subject also reduces the level and/or activity of one or more Th1 and/or Th2 cytokines in the subject without substantially reducing the level and/or activity of innate immune cytokines in the subject. Methods for determining the level and/or activity of cytokines are known in the art.

Accordingly, in another embodiment the present invention provides a method of reducing the level and/or activity of one or more Th1 and/or Th2 antigen-induced cytokines in a subject without substantially inhibiting the level and/or activity of innate immune cytokines in the subject, the method including administering to the subject an effective amount of a polyunsatured fatty acid, or a salt or derivative thereof.

The polyunsaturated fatty acids, or a salt or derivative thereof, of the present invention may be used in the preparation of a medicament. Uses of such medicaments are as described herein.

In another embodiment, the present invention provide use of a polyunsatured fatty acid, or a salt or derivative thereof in the preparation of a medicament for inhibiting the level and/or activity of one or more Th1 and/or Th2 antigen-induced cytokines in a subject without substantially inhibiting the level and/or activity of innate immune cytokines in the subject.

In another embodiment, the administration of the polyunsaturated fatty acids, or a salt or derivative thereof, to the subject inhibits antigen-induced inflammation in the subject.

Accordingly, in another embodiment the present invention provides a method of preventing and/or treating antigen-induced inflammation in a subject, the method including administering to the subject an effective amount of a polyunsaturated fatty acid, or a salt or derivative thereof.

In another embodiment, the present invention provides use of a polyunsaturated fatty acid, or a salt or derivative thereof, in the preparation of a medicament for preventing and/or treating antigen induced inflammation in a subject.

Methods for determining the extent of antigen-induced inflammation are known in the art.

In another embodiment, the administration of the polyunsaturated fatty acids, or a salt or derivative thereof, to the subject increases the level and/or activity of T suppressor cells in the subject.

Accordingly, in another embodiment the present invention provides a method of increasing the level and/or activity of T suppressor cells in a subject, the method including administering to the subject an effective amount of a polyunsaturated fatty acid, or a salt or derivative thereof.

In another embodiment the present invention provides use of a polyunsaturated fatty acid, or a salt or derivative thereof, in the preparation of a medicament for increasing the level and/or activity of T suppressor cells in a subject.

Methods for determining the level and/or activity of T suppressor cells are known in the art.

In another embodiment, the administration of the polyunsaturated fatty acids, or a salt or derivative thereof, to the subject promotes immunosuppressive activity in the subject, including increasing the period and/or level of immunosuppressive activity in the subject.

Accordingly, in another embodiment the present invention provides a method of increasing the period, and/or level of immunosuppressive activity in a subject, the method including administering to the subject an effective amount of a polyunsaturated fatty acid, or a salt or derivative thereof.

In one embodiment, the immunosuppressive activity is suppression of immune activity against one or more antigens without substantial suppression of global immune activity.

In another embodiment, the present invention provides use of a polyunsaturated fatty acid, or a salt or derivative thereof, in the preparation of a medicament for increasing the period and/or level of immunosuppressive activity in a subject.

Methods for determining the period and/or level of immunosuppressive activity are known in the art.

In another embodiment, the administration of the polyunsaturated fatty acids, or a salt or derivative thereof, to the subject increases the immunological tolerance in the subject, including the period and/or level of immunological tolerance.

Accordingly, in another embodiment the present invention provides a method of increasing immunological tolerance in a subject, the method including administering to the subject an effective amount of a polyunsaturated fatty acid, or a salt or derivative thereof.

In one embodiment, the increase in immunological tolerance in the subject is an increase in tolerance to one or more antigens without a substantial increase in global immunological tolerance.

In another embodiment the present invention provides use of a polyunsaturated fatty acid, or a salt or derivative thereof, in the preparation of a medicament for increasing immunological tolerance in a subject.

Methods for determining the period and/or level of immunological tolerance are known in the art.

In another embodiment, the polyunsaturated fatty acids, or a salt or derivative thereof, may be used to promote anergy of T cells.

Accordingly, in another embodiment the present invention provides a method of promoting T cell anergy in a subject, the method including administering to the subject an effective amount of a polyunsaturated fatty acid, or a salt or derivative thereof.

In another embodiment, the present invention provides use of a polyunsaturated fatty acid, or a salt or derivative thereof, in the preparation of a medicament for promoting T cell anergy in a subject.

Methods for determining the level of T cell anergy are known in the art.

It will also be appreciated that the use of the polyunsaturated compounds in the present invention also includes exposing T cells in vivo, ex vivo and/or in vitro to the polyunsaturated compound.

Thus the present invention also contemplates a method of inhibiting antigen dependent proliferation of T cells without substantially inhibiting mitogen dependent proliferation of T cells, the method including exposing the T cells to the polyunsaturated fatty acids, or a salt or derivative thereof, of the present invention.

For example, T cells isolated from a subject by a method known in the art may be treated ex vivo with the polyunsaturated compound and then introduced back into the subject. One suitable application is to isolate T cells from the subject, freeze the cells, expose them to the polyunsaturated fatty acid, or a salt or derivative thereof, and then introduce them back into the subject at the time of disease to suppress inflammation. Alternatively, the cells could be treated prior to freezing.

Accordingly, in another embodiment the present invention provides exposing T cells isolated from a subject to an effective amount of a polyunsaturated fatty acid, or a salt thereof; and introducing the T cells so exposed back into the subject, to produce the various effects of the polyunsaturated fatty acids previously described herein

In another embodiment the present invention also provide use of T-cells isolated from a subject and treated with a polyunsaturated fatty acid, or a salt or derivative thereof, in the preparation of a medicament for producing the various effects of the polyunsaturated fatty acids previously described herein

T cells from a subject may also be cultured in vitro in the presence of the polyunsaturated compound to generate T suppressor cells, which may or may not be further purified, and then re-injected the cells back into the subject.

A suitable method for exposure of the T cells/T suppressor cells to the polyunsaturated fatty acids, or a slat or derivative thereof, may be chosen.

In a similar fashion, the exposure of T cells in vivo, ex vivo and/or in vitro may be used to produce the various effects of the polyunsaturated fatty acids previously described herein, for example: (i) inhibition of Th1 and Th2 cytokine production (the adaptive immune system) but not cytokines formed by the innate immune system; (ii) reduction in antigen induced inflammation; (iii) promotion of the generation of T suppressor/regulatory cells; (iv) promotion of the generation of long lasting immunosuppressive activity; (v) promotion of immunological tolerance; and (vi) promoting T cell anergy.

For example, in another embodiment the present invention a method of increasing immunosuppressive activity in a subject, the method including:
exposing T cells isolated from a subject to an effective amount of a polyunsaturated fatty acid, or a salt thereof; and
introducing the T cells so exposed back into the subject.

In another example, use of T-cells isolated from a subject and treated with a polyunsaturated fatty acid, or a salt or derivative thereof, may be sued in the preparation of a medicament for increasing immunosuppressive activity in the subject when introduced into back into the subject.

In the case of administration of the polyunsaturated fatty acids, or a salt or derivative thereof, to the subject in the various embodiments of the present invention, a suitable method of administration known in the art may be used.

For example, the polyunsaturated fatty acids, or a salt or derivative thereof, of the present invention may be delivered to the desired site of action directly (eg by direct injection), or be delivered by administration of the compound to the subject (eg oral administration).

The polyunsaturated fatty acids, or a salt or derivative thereof, of the present invention will typically be formulated into a suitable composition or medicament for the desired route and mode of delivery or administration.

For example, the polyunsaturated fatty acids, or a salt or derivative thereof, for use in the various embodiments of the present invention may be admixed with a pharmaceutically acceptable solvent, carrier or excipient, and which is typically inert. A pharmaceutical carrier can be any compatible non-toxic substance suitable for delivery of the agent to a subject.

The preparation of pharmaceutical compositions is known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa.

Examples of pharmaceutically acceptable additives include pharmaceutically acceptable salts, amino acids, polypeptides, polymers, solvents, buffers, excipients, preservatives and bulking agents, taking into consideration the particular physical, microbiological and chemical characteristics of the compound to be administered.

The effective amount of the polyunsaturated compound to be delivered is not particularly limited, so long as it is within such an amount and in such a form that generally exhibits a useful or therapeutic effect. The term “effective amount” is the quantity which when delivered or administered, improves the prognosis of the subject. The amount to be delivered will depend on the particular characteristics of the condition being treated, the mode of delivery, and the characteristics of the subject, such as general health, other diseases, age, sex, genotype, body weight and tolerance to drugs. A person skilled in the art will be able to determine appropriate dosages depending on these and other factors.

In this regard, it has been found in the present studies that the polyunsaturated fatty acids, or a salt or derivative thereof, are unexpectedly effective at doses less than 10 mg/kg of body weight.

Thus, the various effects of the polyunsaturated fatty acids, or a salt or derivative thereof, as previously described herein may be produced by administering the compounds at a dose of less than 10 mg/kg body weight.

Such effects include for example (i) inhibition of Th1 and Th2 cytokine production (the adaptive immune system) but not cytokines formed by the innate immune system; (ii) reduction in antigen induced inflammation; (iii) promotion of the generation of T suppressor/regulatory cells; (iv) promotion of the generation of long lasting immunosuppressive activity; (v) promotion of immunological tolerance; and (vi) promoting T cell anergy.

For example, in one embodiment there is provided a method of inhibiting antigen-dependent proliferation of T-cells in a subject, the method including administering to the subject a polyunsaturated fatty acid, or a salt or derivative thereof, at a concentration of less than 10 mg/kg body weight.

In another embodiment, there is provided a method of preventing and/or treating a T-cell mediated disease, condition or state in a subject, the method including administering to the subject a polyunsaturated fatty acid, or a salt or derivative thereof, at a concentration of less than 10 mg/kg body weight.

In another embodiment, there is provided a method of reducing the level and/or activity of one or more Th1 and/or Th2 antigen-induced cytokines in a subject, the method including administering to the subject a polyunsaturated fatty acid, or a salt or derivative thereof, at a concentration of less than 10 mg/kg body weight.

In another embodiment, there is provided a method of preventing and/or treating an auto-immune disease in a subject, the method including administering to the subject a polyunsaturated fatty acid, or a salt or derivative thereof, at a concentration of less than 10 mg/kg body weight.

A pharmaceutical composition including a low dose of the polyunsaturated fatty acid, or a salt or derivative thereof, is also provided.

Accordingly, in another embodiment there is provided a pharmaceutical dosage form for administration to a subject, the dosage form including less than 10 mg/kg body weight of a polyunsaturated fatty acid, or a salt or derivative thereof.

Typical ranges of the polyunsaturated fatty acids, or a salt or derivative thereof, include about 0.05 mg/kg to 5 mg/kg body weight, such as 0.5 mg/kg to 5 mg/kg body weight or 0.05 to 0.5 mg/kg bodyweight. Depending upon the compound, other ranges include 0.01 mg/kg to 5 mg/kg body weight, (such as 0.1 to 5 mg/kg, 0.1 to 1 mg/kg, 0.05 to 1 mg/kg, 1 to 5 mg/kg bodyweight).

In another embodiment, the effective amount of the polyunsaturated fatty acid, or a salt or derivative thereof, is defined by the concentration that the compounds are exposed to T cells.

In one embodiment, the concentration of the compounds exposed to a T cell is equal to or less than 25 μM. In one specific embodiment, the concentration of the compounds exposed to a T cell is equal to or less than than 10 μM. In a further embodiment, the concentration is 2 μM or lower. Suitable ranges include 0.1 to 10 μM, such as 1 to 10 μM.

In the case of subject, these levels also represent the concentration of the compounds in the blood/serum. Accordingly, in one embodiment the effective amount of the blood concentrations of the compounds is equal to or less than than 25 μM. In one specific embodiment, the concentration of the compounds is equal to or less than than 10 μM. In a further embodiment, the concentration is 2 μM or lower Suitable ranges include 0.1 to 10 μM, such as 1 to 10 μM.

Accordingly, there is also provides a pharmaceutical composition including an amount of a polyunsaturated fatty acid, or a salt or derivative thereof, wherein the amount of the polyunsaturated fatty acid, or a salt or derivative thereof, in the composition provides a blood concentration as described above when administered to a subject.

It has also been unexpectedly found in the present studies that the administration of the polyunsaturated fatty acids, or a salt or derivative thereof produces long last effects even after a single administration.

For example, a single administration of the polyunsaturated compounds produces long last effects after 1, 2 and at least up to 6 days after administration.

Accordingly, in one embodiment the administration of the polyunsaturated fatty acids, or a salt or derivative thereof, includes a single administration.

In another embodiment, the administration of the polyunsaturated fatty acids, or a salt or derivative thereof, includes recurrent administration greater than every 6 days.

For example it is envisaged that in some case a single administration may all that is required under some circumstances. Under other circumstances, a recurrent administration every week or greater may be sufficient. Under other circumstances a recurrent administration every month or greater may be sufficient.

The present invention therefore contemplates treatment regimes based on the above.

The actual dosage form, frequency and amount of dose will depend on the mode and route of delivery or administration.

In one embodiment, the administration of the polyunsaturated compound to the subject includes recurrent administration of the polyunsaturated fatty acid to the subject greater than every 6 days.

For example, effective amounts of the polyunsaturated compound of the present invention typically result in the administration of the ranges discussed previously herein every week.

Administration and delivery of the polyunsaturated fatty acids, or a salt or derivative thereof may be, for example, by intravenous, intraperitoneal, subcutaneous, intramuscular, oral, or topical route, or by direct injection into the desired site of action. The mode and route of administration in most cases will depend on the type of disease, condition or state being treated.

In the current studies it has been found that the effects of the compounds are independent of the route of administration.

As described above, the administration of the composition of a polyunsaturated fatty acid, or a salt, or derivative thereof, may also include the use of one or more pharmaceutically acceptable additives, including pharmaceutically acceptable salts, amino acids, polypeptides, polymers, solvents, buffers, excipients, preservatives and bulking agents, taking into consideration the particular physical, microbiological and chemical characteristics of the compound to be administered.

For example, the compounds can be prepared into a variety of pharmaceutical acceptable compositions in the form of, e.g., an aqueous solution, an oily preparation, a fatty emulsion, an emulsion, a lyophilised powder for reconstitution, etc. and can be administered as a sterile and pyrogen free intramuscular or subcutaneous injection or as injection to an organ, or as an embedded preparation or as a transmucosal preparation through nasal cavity, rectum, uterus, vagina, lung, etc. The composition may be administered in the form of oral preparations (for example solid preparations such as tablets, caplets, capsules, granules or powders; liquid preparations such as syrup, emulsions, dispersions or suspensions).

Compositions containing the compound may also contain one or more pharmaceutically acceptable preservative, buffering agent, diluent, stabiliser, chelating agent, viscosity-enhancing agent, dispersing agent, pH controller, solubility modifying agent or isotonic agent. These excipients are well known to those skilled in the art.

Examples of suitable preservatives are benzoic acid esters of para-hydroxybenzoic acid, propylene glycol, phenols, phenylethyl alcohol or benzyl alcohol. Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, and chelating agents, such as disodium edetate. Examples of suitable viscosity enhancing agents, suspending or dispersing agents are substituted cellulose ethers, substituted cellulose esters, polyvinyl alcohol, polyvinylpyrrolidone, polyethylene glycols, carbomer, polyoxypropylene glycols, sorbitan monooleate, sorbitan sesquioleate, polyoxyethylene hydrogenated castor oil 60.

Examples of suitable pH controllers include hydrochloric acid, sodium hydroxide, buffers and the like. Examples of suitable isotonic agents are glucose, D-sorbitol or D-mannitol, sodium chloride.

The administration of the compounds may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, glidant, anti-adherent, binder, flavourant or sweetener, taking into account the physical, chemical and microbiological properties of the compound being administered.

For these purposes, the composition may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile, pyrogen free injectable form (solution, suspension or emulsion, which may have been reconstituted prior to use) which is usually isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing or wetting agents and suspending agents. The sterile injectable forms may also be sterile injectable solutions or suspensions in non-toxic parenterally acceptable diluents or solvents, for example, as solutions in 1,3-butanediol. Among the pharmaceutically acceptable vehicles and solvents that may be employed are water, ethanol, glycerol, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

In addition, the composition containing the compounds of the present invention may be in a form to be reconstituted prior to administration. Examples include lyophilization, spray drying and the like to produce a suitable solid form for reconstitution with a pharmaceutically acceptable solvent prior to administration.

Compositions may include one or more buffer, bulking agent, isotonic agent and cryoprotectant and lyoprotectant. Examples of excipients include, phosphate salts, citric acid, non-reducing sugars such as sucrose or trehalose, polyhydroxy alcohols, amino acids, methylamines, and lyotropic salts are generally used in place of reducing sugars such as maltose or lactose.

In one embodiment, the administration of the polyunsaturated fatty acid, or a salt or derivative thereof, is by oral administration.

When administered orally, the compounds will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include excipients such as lactose, dextrose, sucrose, sorbitol, mannitol, starches, gum acacia, calcium phosphate, mineral oil, cocoa butter, oil of theobroma, alginates, tragacanth, gelatin, syrup, substituted cellulose ethers, polyoxyethylene sorbitan monolaurate, methyl hydroxybenzoate, propyl hydroxybenzoate, talc, magnesium stearate, and the like.

A tablet may be made by compressing or moulding the compound optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the compound may also utilize controlled release technology.

The compound may also be administered as a sustained-release pharmaceutical. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the compound may be incorporated into a hydrophobic polymer matrix for controlled release over a period of days. The composition of the invention may then be moulded into a solid implant, or externally applied patch, suitable for providing efficacious concentrations of the polyunsaturated fatty acid over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be moulded into a solid implant suitable for providing efficacious concentrations of the polyunsaturated fatty acid over a prolonged period of time without the need for frequent re-dosing. The polyunsaturated fatty acids can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

For topical administration, the composition may be in the form of a solution, spray, lotion, cream (for example a non-ionic cream), gel, paste or ointment. Alternatively, the composition may be delivered via a liposome, nanosome, ribosome, or nutri-diffuser vehicle.

The present invention may also be used for screening compounds for their ability to inhibit antigen-dependent proliferation of sensitised T cells.

Accordingly, in another embodiment the present invention provides a method of identifying an agent that inhibits antigen dependent proliferation of T cells, the method including:
providing a test compound;
determining the ability of the test compound to inhibit antigen dependent proliferation of T cells without substantially inhibiting mitogen dependent proliferation of T cells; and
identifying the test compound as an agent that inhibits antigen-dependent proliferation of T cell.

Compounds so identified are suitable for use in the various aspects of the current invention.

The present studies also indicate that a sensitised T cell is driven towards immunoresponsiveness or anergy dependent on the balance of intracellular signaling pathways, and in particular the PKC→ERK1/ERK2 signalling pathway.

Accordingly, in one embodiment, there is provided a method of modulating responsiveness of a sensitised T cell to an antigen, the method including modulating activity of one or more signalling pathways in the T cell.

In one embodiment, there is provided a method of modulating T cell anergy by modulating the activity of one or more signalling pathways in the T cell.

In one embodiment, the signalling pathway is the ERK1/2 signalling pathway.

In another embodiment there is provided a method of modulating T cell anergy by modulating the activity of the ERK1/2 signalling pathway.

For example, unresponsiveness of T cells to an antigen may be promoted by inhibiting the activity of the pathway. This may be important under conditions where lack a response is important, such as auto-immune diseases, and other T-cell mediated diseases as discussed previously herein.

In one embodiment there is provided a method of promoting T cell anergy in a subject, the method including administering to the subject an agent that inhibits the activity of the ERK1/2 signalling pathway in a T cell.

In another embodiment, there is provided a method of increasing the period and/or level of immunosuppressive activity in a subject, the method including administering to the subject an effective amount of an agent that inhibits the activity of the ERK1/2 signalling pathway in a T cell.

In another embodiment, there is provided a method of increasing immunological tolerance in a subject, the method including administering to the subject an effective amount of an agent that inhibits the activity of the ERK1/2 signalling pathway in a T cell.

Alternatively, responsiveness of T cells to a particular antigen may be promoted by promoting the activity of the ERK1/2 signalling pathway. This may be important under situations in which an improved response to an antigen is desired, such as in the case of cancer.

Agents that modulate T cell responsiveness may also be identified by identifying agents that modulate the activity of the ERK1/2 signalling pathway.

Accordingly, in another embodiment there is provided a method of identifying an agent that modulates T cell responsiveness or anergy, the method including identifying an agent that modulates the activity of the ERK1/2 signalling pathway in a T cell.

In one embodiment, there is provided a method of identifying an agent that promotes T cell anergy, the method including identifying an agent that inhibits the activity of the ERK1/2 signalling pathway in a T cell, wherein an agent that inhibits the ERK1/2 signalling pathway is a candidate agent for promoting T cell anergy.

Modulating the immunosuppressive activity of a subject may also be achieved by modulating the activity of one or more signalling pathways in a T cell.

In one embodiment, there is provided a method of modulating immunosuppressive activity in a subject, the method including administering to the subject an effective amount of an agent that modulated the activity of one more signalling pathways in a T cell.

In one embodiment, the signalling pathway is the ERK1/2 signalling pathway.

In one embodiment, there is provided a method of increasing the period and/or level of immunosuppressive activity in a subject, the method including administering to the subject an effective amount of an agent that inhibits the ERK1/2 signalling pathway in a T cell.

In another embodiment, there is provided a method of increasing immunological tolerance in a subject, the method including administering to the subject an effective amount of an agent that inhibits the ERK1/2 signalling pathway in a T cell.

The identification of agents that modulate T cell anergy may also be achieved by identifying agents that modulate the activity of one or more signalling pathways in a T cell.

In one embodiment, there is provided a method of identifying an agent that promotes T cell anergy, the method including identifying an agent that inhibits the ERK1/2 signalling pathway in a T cell, wherein an agent that inhibits the ERK1/2 signalling pathway is a candidate agent for promoting T cell anergy.

### DESCRIPTION OF SPECIFIC EMBODIMENTS

Reference will now be made to experiments that embody the above general principles of the present invention. However, it is to be understood that the following description is not to limit the generality of the above description.

### Example 1

### Preparation of β-oxa-21:3n-3/β-thia-21:3n-3 and Antibodies

β-oxa-21:3n-3 and β-thia-21:3n-3 were synthesized as previously described to approximately 98% purity and stored in aliquots in chloroform at −70° C. in anhydrous nitrogen-sealed glass containers. The preparations were brought to room temperature; desired amounts were transferred to glass tubes, and solvent was evaporated using anhydrous nitrogen. Whole mouse serum (vehicle) was added and mixed gently to dissolve the fatty acids.

Unless otherwise indicated all antibodies were purchased from BD pharmingen (La Jolla, Calif.).

### Example 2

### Immunization of Mice, Administration of β-oxa-21:3n-3/β-thia-21:3n-3 and Ex-Vivo T Cell Proliferation

6-7 weeks old Balb/c mice were injected subcutaneously with 100 μl of 10% sheep red blood cells (IMVS, Inc., Adelaide, Australia) After 5 days 100 ul of β-oxa-21:3n-3 at indicated amounts were injected intraperitoneally, intravenously or gavaged orally. In another experimental set up mice were given β-tha-21:3n-3 i.p. In a third approach mice were infected subcutaneously with 100 μl of Tetanus Toxoid (2,500 Lf/ml). Then 7 days later were given β-oxa-21:3n-3. At indicated time points mice were sacrificed and spleens were removed. Single cell suspensions were prepared and white blood cells (WBC) were enriched by passing through Ficoll-Paque Plus (GE Healthcare). Cells were washed with RPMI-1640 containing 10% fetal bovine serum, 10 mM Hepes, antibiotics and glutamine (RPMI-10) and counted using an automated Cell-Dyne 3500R (Abbott, Inc. MA) instrument. Cells were viable (>98%) as judged by trypan blue exclusion. (β-oxa-21:3n-3)- or vehicle-treated mouse samples (200,000/well) were mixed either with SRBC (600,000/well) with 100 μl of Tetanus toxoid (50 Lf/ml) or phytohaemaglutinin (PHA) (2 μg/ml) or with PHA plus phorbol myristate acetate (PMA) (10 ng/ml) or with PMA plus calcium ionophore A23187 (1 mM). Cells were cultured in 96 well U bottom plates (Nunc, Inc. Denmark) for 3 days (except for TT −5 days) at 37° C. under humidified air containing 5% CO₂. At some instances 50 μl of supernatants were transferred to a new plate and frozen immediately at −70° C. to be used for cytokine measurements. Thymidine (50 μl, 1 μCi/well) was added at final 16 hours of culture. Cells were harvested and thymidine incorporation was determined using a beta scintillation counter.

### Example 3

### Co-Culture Experiment

In co-culture experiments negative selection purification systems were utilized to ensure isolation of untouched cells to avoid side effects on cell viability and activity. Therefore, cells that broke through anti-MHC class II-conjugated beads were used as enriched T cell fractions (depleted of MHC class II cells) and those that broke through the anti-Thy1.2-conjugated beads were used as enriched antigen presenting cell fractions (depleted of T cells). For accuracy in cell counts and cell viability, the enriched cells were also inspected by hemocytometer and then counted using Cell-Dyne 3500R (ABBOTT, Inc. MA) and then co-cultured.

### Example 3

### Flow Cytometry

Data acquisition was performed using a BD Immunocytometry Systems four-color FACScalibur (Mountain View, Calif.), and the acquired data were analyzed using FloJo software (Tree Star Inc., Palo Alto, Calif.).

### Example 4

### Determination of IL-2, IL-4, IL-5, IFNγ and TNFα in the Blood and Spleen

Mice were injected subcutaneously with SRBC. After 5 days β-oxa-21:3n-3 (80 mg/kg) or vehicle (whole mouse serum) were injected intraperitoneally. 2 hours later SRBC were injected intravenously. The blood was collected from orbital plexus at 6 hours post intravenous SRBC injection. Serum was separated and frozen immediately at −70° C. The levels of cytokine were measured using BD mouse CBA kits using a BD Array instrument (BD, La Jolla, Calif.).

### Example 5

### Human Lymphocyte Proliferation Assay

Peripheral blood mononuclear cells were purified from the blood of normal volunteers using hypagn-ficoll density gradient separation. In the assay 100 μl of PBM (treated or untreated) at a concentration of 2×10⁶/ml were stimulated by the addition of 100 μl of PHA (μg/ml) or 1000 μl 2 μg/ml TT in microtitre plates. The cultures contained 5% human blood group AB serum. The cells were incubated at 37° C. in 5% CO₂-air and high humidity for 72 h or 5 days (for TT). The cultures were pulsed with 1 μl of 3H-TdR 6 h prior to harvest. The amount of radioactivity incorporated was determined to assess the degree of lymphoproliferation.

### Example 6

### Assessment of β-oxa-21:3n-3 Incorporation into Phospholipids and DAG

T cells, purity of >98% CD3⁺ cells by FACScan analysis, were purified from the peripheral blood of healthy volunteers by a combination of density gradient centrifugation and adhesion nylon wool columns. Viability was >99% as determined by trypan blue dye exclusion. T cells (5×10⁷ cells) were treated with β-oxa-21:3n-3 and incubated with or without PHA-PMA (see above) for 30 min at 37° C. After centrifugation (600×5 min), the pellets were resuspended in water. Lipids were extracted with chloroform:methanol:acetic acid (1:2:0.02, v/v/v). After 60 min, the phases were partitioned by the addition of chloroform:water (1:1), centrifuged (1500 g×5 min) and the top phase was removed and re-extracted. The bottom phases were combined, dried and the samples were processed as described above.

### Example 7

### Suppression of Ex-Vivo Antigen-Induced Lymphoproliferation

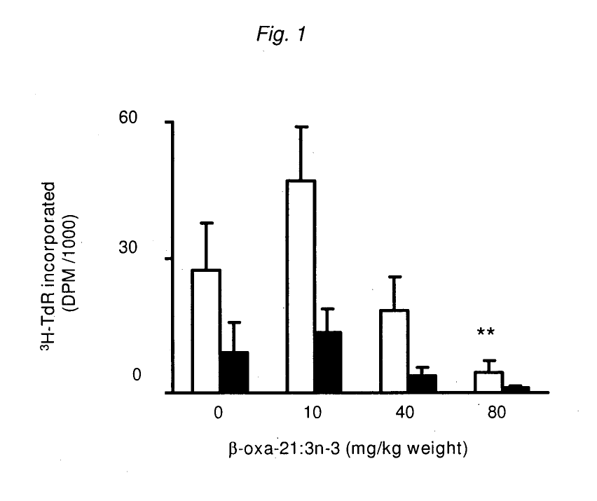

In previous studies intraperitoneal (IP) administration of β-oxa-21:3n-3 resulted in the inhibition of delayed-type hypersensitivity to SRBC antigen on mouse hind paws. To dissect the mechanisms by which β-oxa-21:3n-3 exerts immunoregulatory functions, we have elected to study β-oxa-21:3n-3 role(s) in antigen-dependent splenocyte proliferation, ex vivo. A single IP injection of β-oxa-21:3n-3 (dissolved and delivered in 7% dimethyl sulfoxide (DMSO) for 2 h (SRBC pre-sensitized mice resulted in a dose-dependent inhibition of antigen-dependent proliferation of splenic lymphocytes, ex-vivo (FIG. 1).

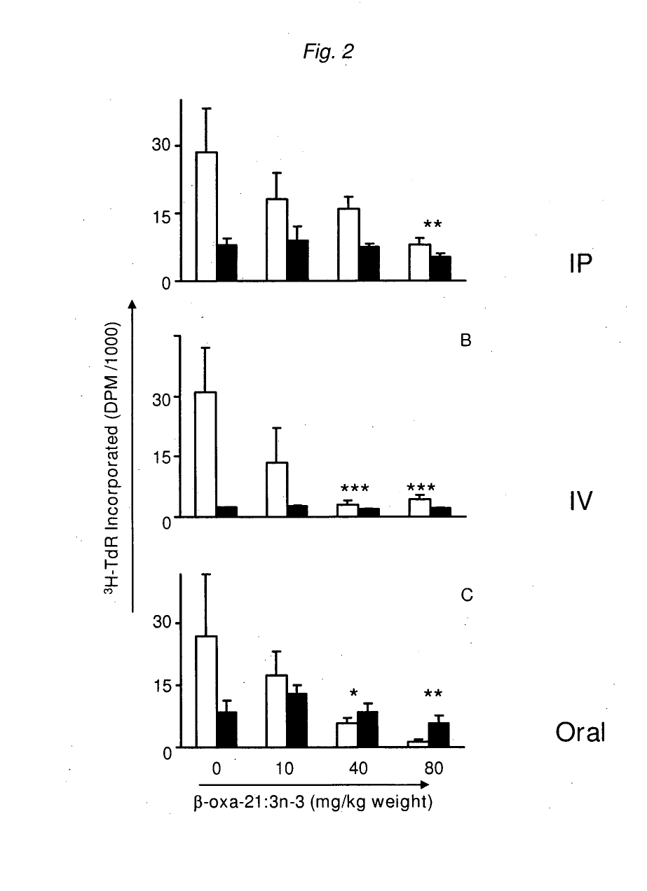

In order to decrease side effect of chemicals such as DMSO that may activate macrophages during intraperitoneal injections, the β-oxa-21:3n-3 was delivered in syngeneic mouse serum. The results presented in FIG. 2A show that, IP administration of β-oxa-21:3n-3 also resulted in a similar dose-dependent inhibition of splenocyte proliferation. This inhibitory effect was even more pronounced upon intravenous administration of β-oxa-21:3n-3 (FIG. 2B). Orally administered β-oxa-21:3n-3 also significantly depressed the antigen-dependent proliferation of splenocytes (FIG. 2C).

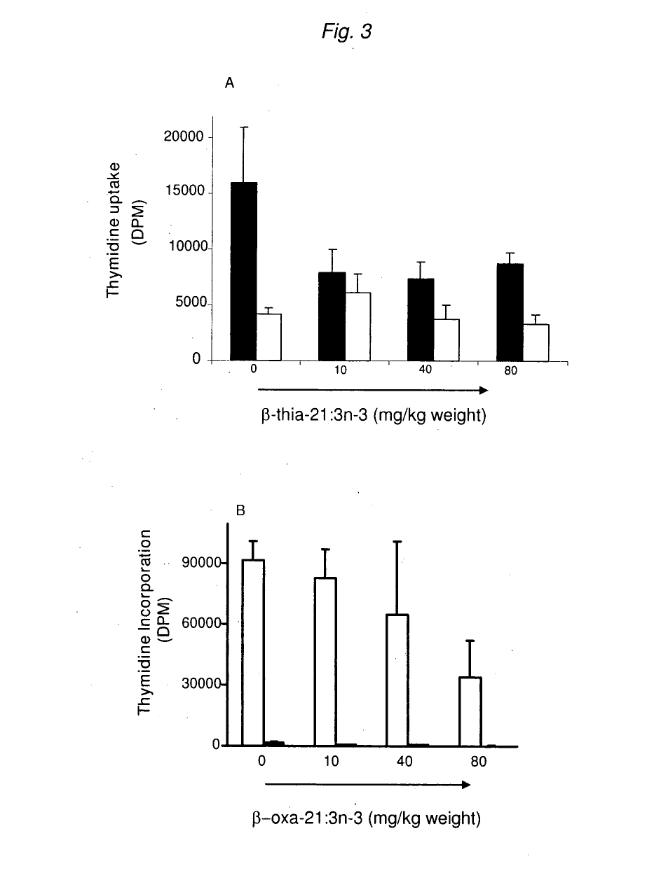

To see if this effect was specific to the oxygen function in the β-position we examined the effect of using the β-thia derivative, β-thia-21:3n-3. In these experiments mice, were similarly immunized to SRBC and 5 days later the β-thia-21:3n-3 was administered ip in mouse serum. The lymphoproliferative response to SRBC was then determined in ex vivo experiments using splenocytes and measuring lymphoproliferation. The results showed that β-thia-21:3n-3 caused inhibition of the lymphoproliferative response in a dose dependent manner between 10-80 mg/kg (FIG. 3A).

To ascertain that the antigen-induced immunosuppression was not specific to SRBC, we examined the effect of β-oxa-21:3n-3 on the response to tetanus toxoid. In these experiments mice were sensitized to TT subcutaneously. After 7 days the animals received β-oxa-21:3n-3 at doses of 10, 40 and 80 mg/kg i.p. in mouse serum. Two hours later the spleens were removed, spleen cells prepared and tested for lymphoproliferation in response to TT. The results showed that the fatty acid was capable of inhibiting the response to this antigen (FIG. 3B).

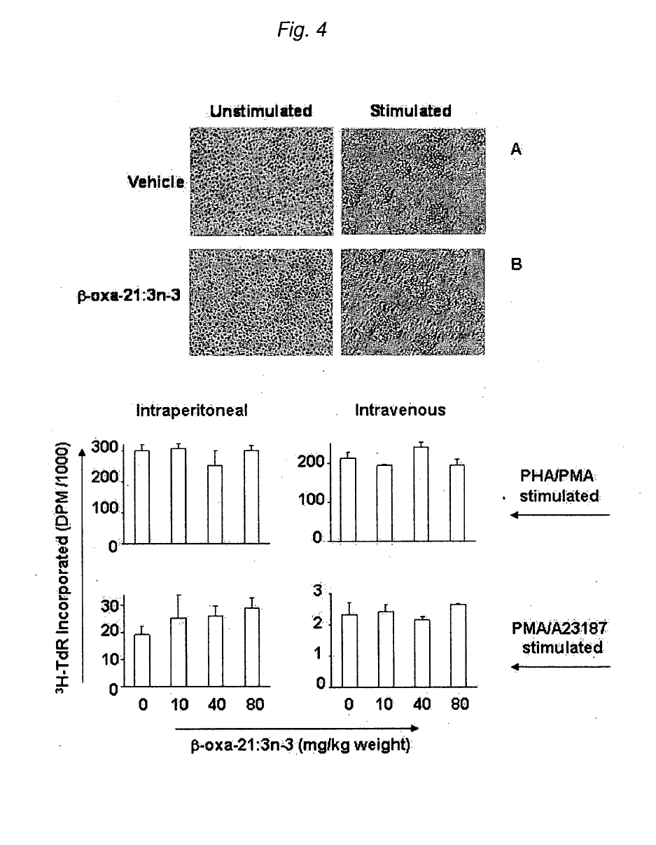

To assess whether or not the immunosuppression observed was restricted to antigen-dependent stimulation, we explored the effect of β-oxa-21:3n-3 on mitogen-induced splenocyte adhesion and proliferation. Mice pre-sensitized to SRBC were given vehicle- or β-oxa-21:3n-3). After 4 h splenic cells were isolated and stimulated with PHA and PMA for 4 h and then examined for splenocyte adherence. The data showed that after 4 h, splenocyte adherence, a phenomenon that occurs prior to proliferation, was not affected by administration of β-oxa-21:3n-3 (FIG. 4A). The cells were cultured for 3 days and the extent of proliferation was assessed by thymidine incorporation. As shown in FIG. 4B) splenocytes prepared from IP- or IV-administered β-oxa-21:3n-3 mice were not affected in their responsiveness to PHA-PMA or PMA-A23187. β-oxa-21:3n-3 administration also did not affect the splenocyte proliferation in response to PHA alone (not shown).

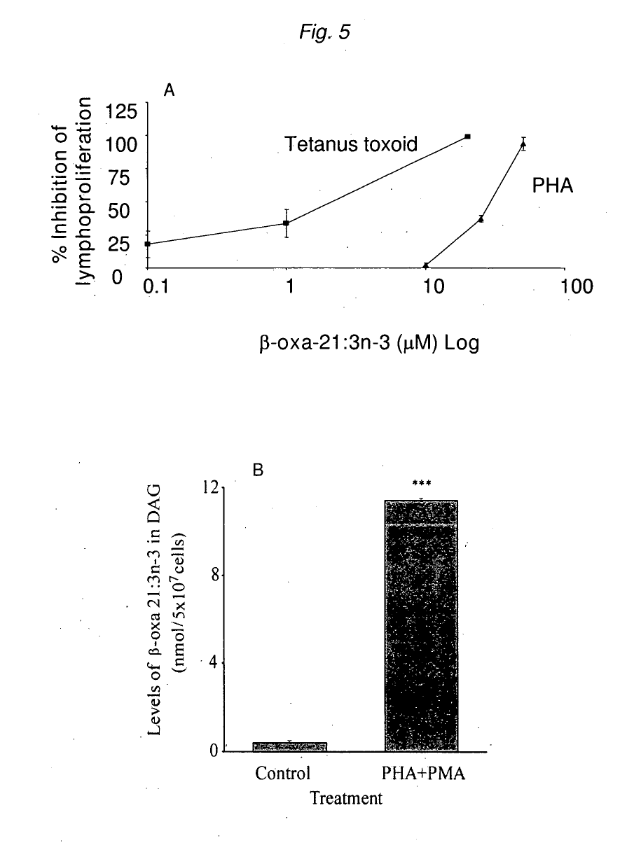

Previously we had demonstrated that β-oxa21:3n-3 inhibited the mitogenic (PHA)-induced lymphoproliferation of human lymphocytes, which stands in contrast to the present results from the ex-vivo experiments. To attempt to resolve this discrepancy, we examined the sensitivity of the antigen-induced (tetanus toxoid) human lymphocyte response in vitro, under the same conditions as those in which the PHA-induced response were inhibited by β-oxa21:3n-3. Human PBMC which were known to show lymphoproliferation in response to TT were pretreated with a range of concentrations of β-oxa21:3n-3 and then stimulated with TT. Proliferation was quantitated after 72 h of incubation of cultures. The results showed that the IC₅₀ of β-oxa21:3n-3 was 2 μM for the inhibition of the antigen-induced response versus 30 μM for the PHA-stimulated response (FIG. 5A). This shows that the antigen-induced response is 15 fold more sensitive than the mitogenic response. Using purified human T cells which were stimulated with PHA-PMA we were able to ascertain that when β-oxa21:3n-3 was added to cells the stimulated cells contained 30 fold more β-oxa-21:3n-3 than resting T cells (FIG. 5B).

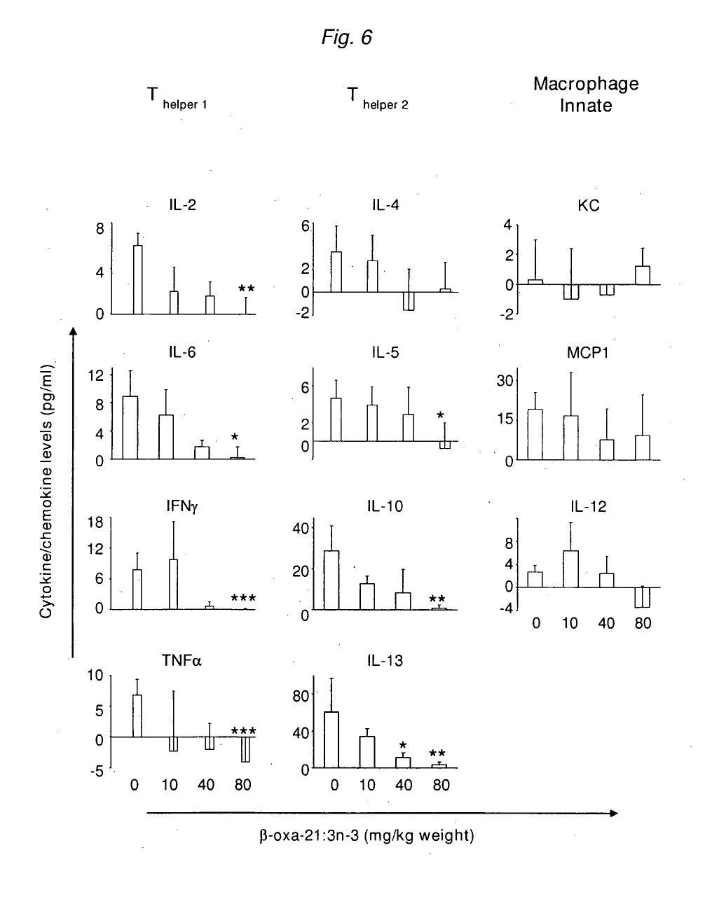

To see if the immunosuppressive action of single β-oxa-21:3n-3 administration was short or long lived, the effect of delaying the spleen cell isolation from mice was examined. As depicted in FIG. 6A, a significant inhibition of splenocyte proliferation occurred even when the ex-vivo SRBC challenge of pre immune mouse splenocytes were performed 1, 2 and 6 days post fatty acid treatment, indicating that the effects of β-oxa-21:3n-3 are long lasting.

To exclude that this effect of β-oxa-21:3n-3 was not due to necrosis or apoptosis of the splenic cells, we examined the viability and composition of live splenic cells from animals treated with this fatty acid, by microscopy and flow cytometry. The results showed that there was no major difference in viable cell numbers (>95% excluded propidium iodide) in spleen cells from (β-oxa-21:3n-3)- or vehicle-treated SRBC-sensitized mice.

### Example 8

### Inhibition of Antigen-Stimulated Th1 and Th2, but not Innate Immune Cytokines Production, Ex-Vivo and In Vivo

The specific and concentration-dependent immunosuppression by β-oxa-21:3n-3 on antigen-dependent, but not mitogen-induced proliferation of splenocytes led us to investigate immune parameters that might be affected by β-oxa-21:3n-3 administration. Thus, the levels of twelve cytokine and chemokines associated with the Th1 or Th2 arm of the immune system as well as those that are engaged in innate immunity were measured in both ex-vivo and in vivo experimentation. Splenocytes from β-oxa-21:3n-3- or vehicle-treated animals (pre-sensitized to SRBC) were cultured for 3 days in the absence or presence of SRBC and culture fluids were collected to measure the level of cytokines and chemokines produced. As shown in FIG. 6, administration of β-oxa-21:3n-3 to mice resulted in a concentration-dependent reduction of antigen-stimulated Th1 (e.g. IL-2, IL-6, IFNγ and TNFα) and Th2 (e.g. IL-4, IL-5, IL-10 and IL-13) cytokines but had no significant effects on antigen-induced cytokine levels such as those secreted by macrophages (e.g. MCP-1, KC, IL-12) which are involved in promoting innate immunity against microbial pathogens.

The insights from these cytokine studies prompted us to investigate whether injected β-oxa-21:3n-3 could regulate cytokine levels in vivo. The β-oxa-21:3n-3- or vehicle-administered pre-immune animals were challenged intravenously with SRBC; after 6 h the serum prepared from the mouse blood and cytokine levels measured. The data in Table 2 show that, β-oxa-21:3n-3 caused significant inhibition of the majority of the Th1 and Th2 cytokines as well as the pleotropic physiologic anti-inflammatory cytokine TGFβ, but had no significant effect or slightly enhanced those involved in innate immunity (e.g. MCP-1).

TABLE 1
Effect of β-oxa-21:3n-3 on circulating cytokine levels.
Cytokine/Chemokine conc (pg/ml)

| Th1 | Vehicle | β-oxa-21:3n-3 |
|---|---|---|
| IL-2 | 111 ± 25 | 73 ± 10* |
| IFNγ | 357 ± 15 | 229 ± 40* |
| TNFα | 22 ± 2 | 15 ± 3 |
| IL-6 | 47 ± 10 | 35 ± 8 |
| Th2 | | |
| IL-4 | 7.3 ± 0.8 | 3.5 ± 2* |
| IL-5 | 152 ± 50 | 120 ± 21 |
| IL-10 | 23 ± 3 | 9 ± 5* |
| IL-13 | 11.7 ± 11 | 0 ± 0 |
| Innate | | |
| MCP-1 | 1152 ± 194 | 2039 ± 457* |
| KC | 63 ± 4 | 61 ± 21 |
| IL-12 | 385 ± 89 | 320 ± 145 |
| Physiologic | | |
| TGFβ | (80 ± 6) × 10³ | (52 ± 6) × 10³ |

Notes to Table 1:
β-oxa-21:3n-3 or vehicle were injected intraperitoneally to 5 days SRBC-sensitized mice. After 2 h SRBC was injected intravenously and 6 h later blood was collected from the retro orbital plexus and serum was prepared for measurement of cytokines. The levels of cytokines were determined by ELISA (TGFβ) and BD cytokine array kit (other cytokines).

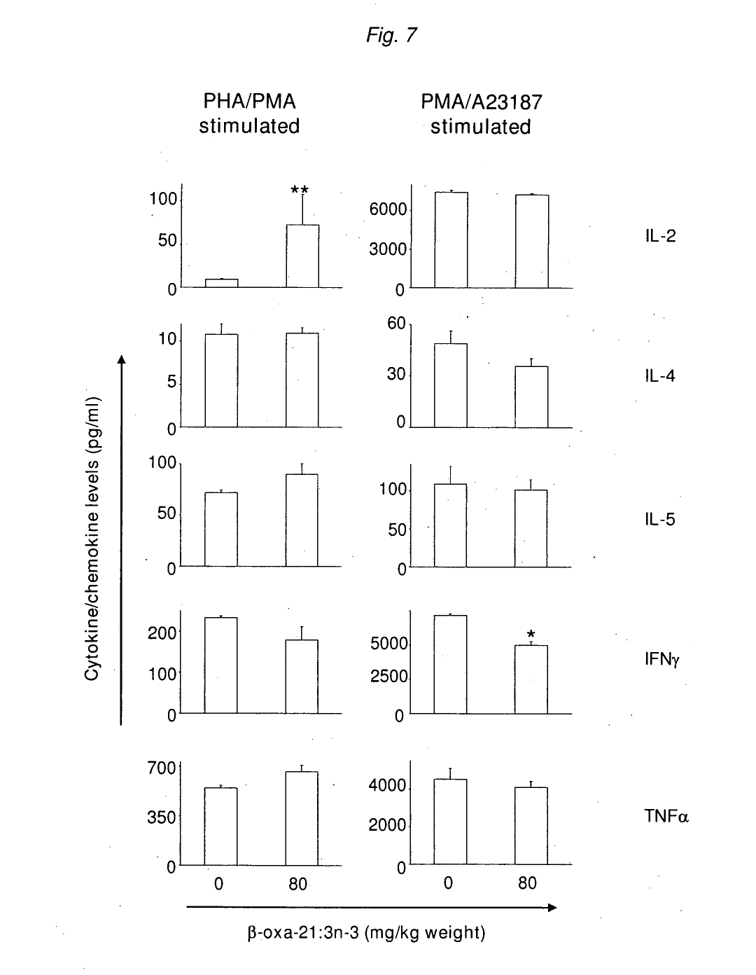

To determine whether or not these effects were due to non-specific suppression of the immune system, we examined the production of Th1 and Th2 cytokines in mitogen-activated mononuclear leukocytes. Splenocytes from (β-oxa-21:3n-3)- or vehicle-administered pre-immune animals were cultured for 3 days in the presence of PHA-PMA or PMA-A23187. The cell culture fluids were collected and cytokines measured. The data show that PHA-PMA-stimulated splenocytes of (β-oxa-21:3n-3)-administered animals secreted higher levels of IL-2 but no substantial increase in the levels of IL-4, IL-5, TNFα and IFNγ could be detected (FIG. 7). Stimulating the cells from β-oxa-21:3n-3-administered animals with PMA-A23187 did not result in any substantial differences in cytokine secretion, apart from a reduction in IFNγ.

### Example 9

### T Cells from β-oxa-21:3n-3 Treated Mice are Unresponsive to Antigen Stimulation

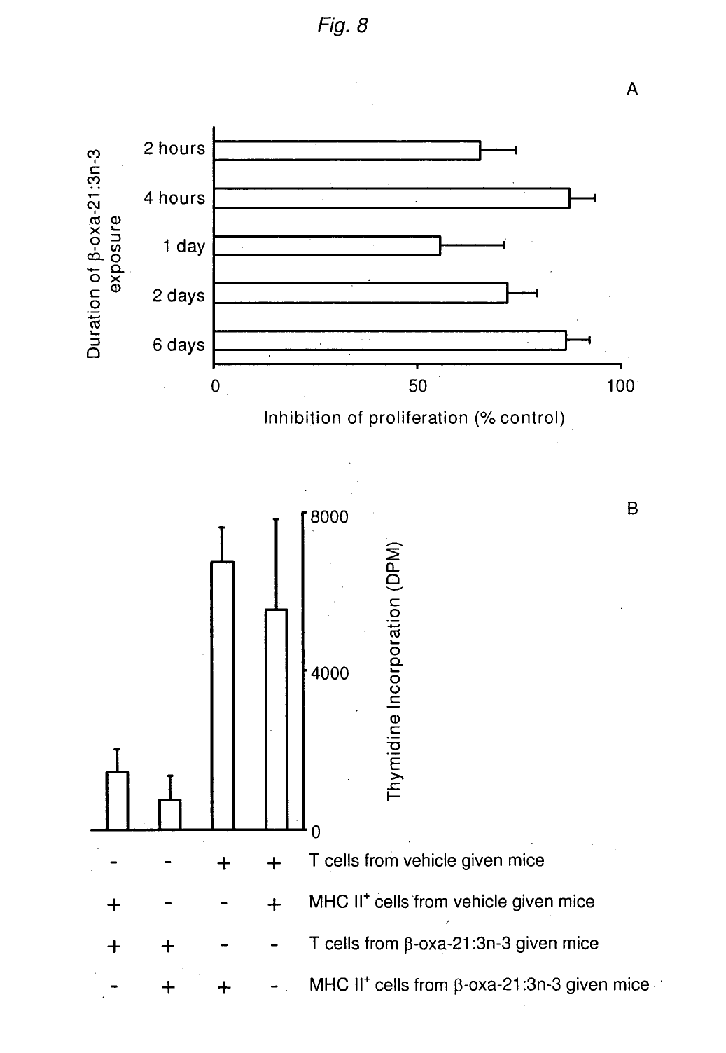

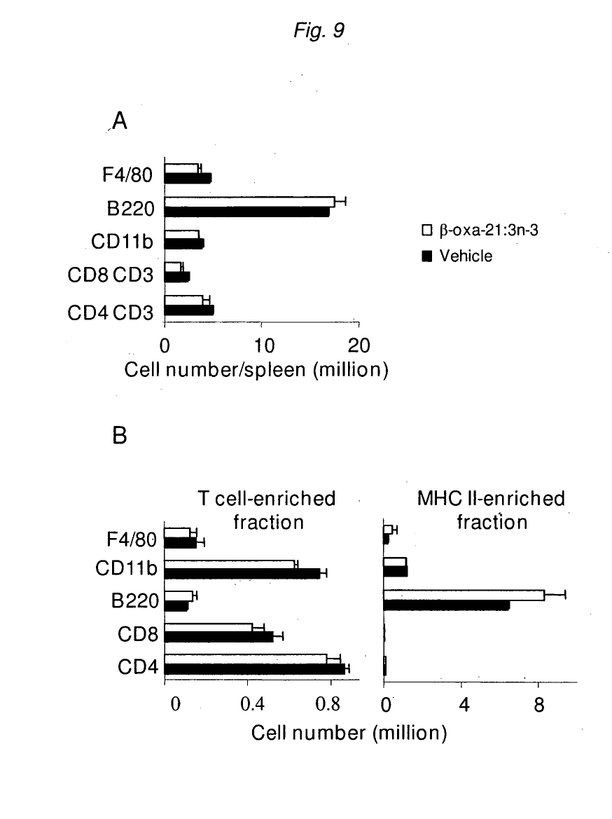

We next examined the cell type that might be affected by the administration of β-oxa-21:3n-3 and/or is responsible for this suppression, namely T cells and antigen presenting cells. Splenic T cells and MHC class II cells from (β-oxa-21:3n-3)- or vehicle-treated animals were enriched using MACS beads (Miltenyi Biotech, Germany). Flow cytometry revealed that MHC class II-enriched fractions contained primarily B220 positive B cells whereas T cell enriched fraction contained CD4 and CD8 positive T cells as well as CD11b positive cells (FIG. 9B). Flow cytometry analysis of cells that were bound to the column revealed the presence of non-specific attachment of F4/80 and CD11b cells (not shown). As depicted in FIG. 8B, splenocyte proliferation was only suppressed when splenic T cells enriched from (β-oxa-21:3n-3)-treated animals were co-cultured with splenic MHC class II cells enriched from either (β-oxa-21:3n-3)- or vehicle-treated animals. In contrast, no suppression occurred when splenic MHC class II cells enriched from (β-oxa-21:3n-3)-treated animals were co-cultured with T cells enriched from either (β-oxa-21:3n-3)- or vehicle-treated animals.

### Discussion

In this study, we discovered that a single administration of β-oxa-21:3n-3 (40-80 mg/kg) to the mice inhibited the ex-vivo antigen-induced, but not mitogen (PHA)-induced, T cell proliferation. This effect was independent of the route of delivery of the fatty acid (i.e. intraperitoneal, intravenous and oral). Importantly the work showed that the synthetic PUFA mimetic β-oxa-21:3n-3 causes the suppression of both Th1 and Th2 type responses only in antigen-dependent fashion, both in in vivo and ex vivo studies. The inhibition of proliferation coincided with down-regulation of both ex-vivo and in vivo Th1 and Th2 cytokine levels. In contrast, β-oxa-21:3n-3 appeared to have no significant inhibitory effect on innate immune and physiologic anti-inflammatory cytokine MCP-1 levels. Gas chromatography and mass spectrometry analysis of extracted lipids from β-oxa-21:3n-3 treated mouse splenocytes did not show any changes in the natural lipid composition of splenocytes, suggesting that β-oxa21:3n-3 has no major effect on lipid metabolism.

Using human peripheral blood lymphocytes or PBMC, we demonstrate for the first time that the antigen versus mitogenic-induced immunosuppression can be distinguished by a substantial β-oxa-21:3n-3 concentration difference. In this regard low concentrations of some fifteen fold less selectively target the antigen-sensitive T cells. This essentially mimics the differential sensitivity seen in the ex-vivo experiments between the specific and non-specific immunoresponsiveness. Co-culture experiments with splenocytes from mice treated with β-oxa-21:3n-3 or vehicle showed that this action of β-oxa-21:3n-3 is exerted at least through splenic T cells. The striking observation, however, was the ability of β-oxa-21:3n-3 to exert this function even 6 days after a single administration, suggesting that the effect is not transient. One explanation is that the β-oxa-21:3n-3 preferentially targets the sensitised T cells through a difference in the amount incorporated between sensitized versus resting T cells. Under the restricted dosaging it is likely that only the sensitized T cells incorporate an adequate amount of β-oxa-21:3n-3 to affect the signaling pathway of PKC→ERK1/ERK2. This is conducive with the concepts that as to whether a T cell is driven towards immunoresponsiveness or anergy dependent on the balance of intracellular signaling pathways⁷.

Finally, it will be appreciated that various modifications and variations of the described methods and compositions of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A method of inhibiting antigen-dependent proliferation of T-cells in a subject without substantially inhibiting mitogen-dependent proliferation of T-cells in the subject, the method including administering to the subject an effective amount of a polyunsaturated fatty acid, or a salt or derivative of a polyunsaturated fatty acid.

2. The method of claim 1, wherein the subject is susceptible to developing a T-cell mediated disease, condition or state.

3. The method of claim 1, wherein the polyunsaturated fatty acid is a polyunsaturated fatty acid including an oxa and/or thia substitution at either or both of the β and γ position of the polyunsaturated fatty acid.

4. The method of claim 1, wherein the derivative of the polyunsaturated fatty acid is a polyunsaturated fatty acid covalently coupled to an amino acid.

5. The method of claim 1, wherein the polyunsaturated fatty acid or the derivative thereof includes one or more substitutions selected from the group consisting of a hydroxyl, a hydroperoxy, a peroxy, and a carboxyalkyl substitution.

6. The method of claim 1, wherein the derivative of the polyunsaturated fatty acid is a polyunsaturated nitroalkene or nitroalkyne.

7. The method of claim 1, wherein the polyunsaturated fatty acid or the derivative thereof contains 16 to 26 carbon atoms.

8. The method of claim 1, wherein the polyunsaturated fatty acid or the derivative thereof includes 3 to 6 carbon double bonds.

9. The method of claim 1, wherein the polyunsaturated fatty acid or derivative thereof is an n-3 to n-6 fatty acid or an n-3 to n-6 nitroalkene or nitroalkyne.

10. The method of claim 1, wherein the effective amount of polyunsaturated fatty acid or the derivative thereof administered to the subject is less than 10 mg/kg bodyweight.

11. The method of claim 1, wherein the administration of the polyunsaturated fatty acid or derivative thereof to the subject includes recurrent administration to the subject greater than every 6 days.

12. The method of claim 1, wherein the administration of the polyunsaturated fatty acid or derivative thereof to the subject reduces the level and/or activity of one or more Th1 and/or Th2 cytokines in the subject without substantially reducing the level and/or activity of innate immune cytokines in the subject.

13. The method of claim 1, wherein the administration of the polyunsaturated fatty acid or derivative thereof to the subject inhibits antigen-induced inflammation in the subject.

14. The method of claim 1, wherein the administration of the polyunsaturated fatty acid or derivative thereof to the subject increases the level and/or activity of T suppressor cells in the subject.

15. The method of claim 1, wherein the administration of the polyunsaturated fatty acid or derivative thereof to the subject increases the period and/or level of immunosuppressive activity in the subject.

16. The method of claim 1, wherein the administration of the polyunsaturated fatty acid or derivative thereof to the subject increases the period and/or level of immunological tolerance in the subject.

17. Use of a polyunsaturated fatty acid, or a salt or a derivative thereof, in the preparation of a medicament for inhibiting antigen-dependent proliferation of T-cells in a subject without substantially inhibiting mitogen-dependent proliferation of T-cells in the subject.

18-102. (canceled)

103. A method of increasing immunosuppressive activity in a subject, the method including: exposing T cells isolated from a subject to an effective amount of a polyunsaturated fatty acid, or a salt thereof; and introducing the T cells so exposed back into the subject.

104-124. (canceled)
